(19) United States
(12) Patent Application Publication
(10) Pub. No.: US 2002/0136732 A1
(43) Pub. Date: Sep. 26, 2002

(54) Compositions comprising carriers and transportable complexes

(76) Inventor: L. L. Houston, Del Mar, CA (US)

Correspondence Address: Andrew E. Granston, FOLEY & LARDNER, 402 W. Broadway, 23rd Floor, San Diego, CA, 92107, US

(21) Appl. No.: 09/839,746

(22) Filed: Apr. 19, 2001

Related U.S. Application Data
(60) Provisional application No. 60/199,423, filed on Apr. 23, 2000.

(51) Int. Cl. A61K 48/00; A61K 39/395; A61K 31/715

(52) U.S. Cl. 424/178.1; 514/44; 514/54

(57) ABSTRACT

The present invention is directed toward compositions comprising one or more transportable complexes and one or more carriers and methods of making and using the same.

### FIELD OF THE INVENTION

The present invention generally relates to compositions, methods of manufacturing and methods of delivering compounds across cell barriers using cell-based transport mechanisms.

### BACKGROUND OF THE INVENTION

The following description of the background of the invention is provided to aid in understanding the invention, but is not admitted to describe or constitute prior art to the invention.

Delivery of compounds, particularly macromolecules, across semi-permeable membranes remains a limitation to drug delivery systems. Furthermore, degradation and inefficient absorption of such compounds delivered by conventional means further reduces the efficacy of those compounds. The ability to utilize alternative delivery pathways, target particular cells and tissues for delivery, improve the retention and absorption of compounds to be delivered, and protect the effective compound during delivery is an ongoing goal of the pharmaceutical and biopharmaceutical industries.

In U.S. Pat. No. 6,042,833, Mostov et al. disclosed a method by which a ligand can bind to a portion of polymeric immunoglobulin receptor (pIgR) and the ligand can be internalized into the cell expressing pIgR.

### SUMMARY OF THE INVENTION

The instant invention is directed to compositions of matter and methods of making and using such compositions.

Thus, a first aspect of the invention concerns compositions. According to the invention, a “composition” refers to a mixture comprising at least one carrier, preferably a physiologically acceptable carrier, and one or more transportable complexes. The term “carrier” defines a chemical compound that does not inhibit or prevent the incorporation of the transportable compound(s) into cells or tissues. A carrier typically is an inert substance that allows an active ingredient to be formulated or compounded into a suitable dosage form (e.g., a pill, a capsule, a gel, a film, a tablet, a microparticle (e.g., a microsphere), a solution etc.). A “physiologically acceptable carrier” is a carrier suitable for use under physiological conditions that does not abrogate (reduce, inhibit, or prevent) the biological activity and properties of the compound. For example, dimethyl sulfoxide (DMSO) is a carrier as it facilitates the uptake of many organic compounds into the cells or tissues of an organism. Preferably, the carrier is a physiologically acceptable carrier, preferably a pharmaceutically or veterinarily acceptable carrier, in which the transportable complex is disposed. A “pharmaceutical composition” refers to a composition wherein the carrier is a pharmaceutically acceptable carrier, while a “veterinary composition” is one wherein the carrier is a veterinarily acceptable carrier. The term “pharmaceutically acceptable carrier” or “veterinarily acceptable carrier” includes any medium or material that is not biologically or otherwise undesirable, i.e., the carrier may be administered to an organism along with a transportable complex, composition or compound without causing undesirable biological effects or interacting in a deleterious manner with the complex or any of its components or the organism. Examples of pharmaceutically acceptable reagents are provided in The United States Pharmacopeia, The National Formulary, United States Pharmacopeial Convention, Inc., Rockville, Md. 1990, hereby incorporated by reference herein into the present application.

The terms “therapeutically effective amount” or “pharmaceutically effective amount” mean an amount sufficient to induce or effectuate a measurable response in the target cell, tissue, or organism. What constitutes a therapeutically effective amount will depend on a variety of factors which the knowledgeable practitioner will take into account in arriving at the desired dosage regimen. The formulation of therapeutic compositions and their subsequent administration is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill in the art can easily determine optimum dosages, dosing methodologies and repetition rates. Optimum dosages may vary depending on the relative potency of individual compounds, and can generally be estimated based on EC₅₀s found to be effective in in vitro and in vivo animal models. In general, dosage is from 0.01 ug to 100 g per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the therapeutic compound is administered in maintenance doses, ranging from 0.01 .ug to 100 g per kg of body weight, once or more daily, to once every 20 years.

A transportable complex of the invention comprises a transportable compound associated with a recognition element for a cell surface transport moiety. A “transportable compound” refers to any compound that can be transported across a cell membrane, preferably by active transport (i.e., a process wherein cellular energy is expended). A number of active transport mechanisms across cell membranes are known in the art. In certain aspects of the invention, the transportable complex, or at least the transportable compound, is moved across or through a cell and released from the cell at a location different from where it entered. For example, in the context of epithelial cells lining the intestine, the cells have a surface presented to the lumen of the intestine, as well as a basolateral surface opposite the lumenal surface. A transportable complex that comes into contact with such a cell can be transported across the cell and it, or at least the transportable compound, can be released from the basolateral surface of the cell. Transportable compounds include small molecules, peptides, polypeptides, nucleic acids, lipids, carbohydrates, and molecules comprising combinations of such molecules, for example, glycoproteins, glycolipids, lipoproteins, etc.

A “small molecule” refers to a synthetic or naturally occurring organic molecule (including synthetic versions of naturally occurring molecules), excluding peptides and nucleic acids, but including compounds such as peptidomimetics, which mimic functional parts of other macromolecules, that has a molecular weight of less than about 5 kilodaltons (kD), preferably less than about 2 kD, even more preferably less than about 1.5 kD. Representative small molecules include chemotherapeutic compounds (e.g., Asparaginase, Bleomycin, Capecitabine, Carboplatin, Cisplatin, Cyclophosphamide, Cytarabine, Dacarbazine, Dactinomycin, Daunorubicin, Dexrazoxane, Docetaxel, Doxorubicin, Etoposide, Floxuridine, Fludarabine, Fluorouracil, Gemcitabine, Hydroxyurea, Idarubicin, Ifosfamide, Iinotecan, Lomustine, Mechlorethamine, Mercaptopurine, Melphalan, Methotrexate, Mitomycin, Mitotane, Mitoxantrone, Paclitaxel, Pamidronate, Pentostatin, Plicamycin, Procarbazine, Rituximab, taxol, taxol derivatives, Teniposide, Thioguanine, Thiotepa, Vinblastine, Vincristine, and Vinorelbine) and other antibiotics (e.g., penicillin, ampicillin, tetracycline, amoxicillin, idoxuridine (e.g., acyclovir, flucytosine, rifampin, naladixic acid/quinolone group (including norfloxacin and ceprafloxacin), polyenes and imidazoles (e.g., nystatin, miconazole, ketoconazole, amphotericin B, and fluconazole), aminoglycosides (e.g., streptomycin, gentamicin, tobramycin, and amikacin), aminocyclitol (e.g., spectinomycin), chloramphenicol, clindamycin, erythromycin, tetracyclines (e.g., doxycycline), penicillins (e.g., penicillin G), aminopenicillins (e.g., amoxicillin, semisynthetic penicillins, e.g., methicillin, nafcillin, and oxacillin, carboxypenicillins (e.g., carbenicillin and ticarcillin), ureidopenicillins (e.g., mezlocillin, azlocillin, and piperacillin), cephalosporins (e.g., cefazolin, cephapirin, cephalexin, cefadroxil, cefoxitin, cefaclor, cefotetan, cefuroxime, cefamandole, cefonocid, moxalactam, cefotaxime, cefoperazone, ceftriaxone, ceftazidime, clavulinic acid, sulbactam, combinations of amoxicillin/clavulinic acid and ampicillin/sulbactam, imipenem, cilistatin, aztreonam, and vancomycin), although any small molecule can be incorporated into a composition according to the invention.

A “peptide” refers to any polymer of two or more amino acids, wherein each amino acid is linked to one or two other amino acids via a peptide bond (—CONH—) formed between the NH₂ and the COOH groups of adjacent amino acids. Preferably, the amino acids are naturally occurring amino acids, particularly α-amino acids of the L-enantiomeric form. However, other amino acids, enantiomeric forms, and amino acid derivatives may be included in a peptide. Peptides include “polypeptides,” which, upon hydrolysis, yield more than two amino acids. Preferred polypeptides include proteins, which typically comprise 50 or more amino acids. Preferred proteins for incorporation into a composition according to the invention as transportable compounds include hormones, cytokines, antibodies, antibody fragments, enzymes, complement components, blood coagulation proteins and soluble receptors. Preferred peptide hormones include insulin, growth hormone, luteinizing hormone, any follicle stimulating hormone, although any peptide or polypeptide can be employed in practicing the invention. Cytokines are proteins involved in signaling between cells during an immune response or involved in an inflammatory response. Lymphokines are a class of cytokines produced by lymphocytes. Representative cytokines include interferons (IFNs; e.g., IFNα, IFNβ, and IFNγ), interleukins (including IL-1 to IL-15), and colony stimulating factors (e.g., those involved in the division and differentiation of bone marrow stem cells and their progeny, for example, stem cell factor (SCF), granulocyte colony stimulating factor (G-CSF), erythropoietin (EPO), granulocyte macrophage colony stimulating factor (GM-SCF)), fibroblast growth factors (e.g. FGF1 & FGF2), PDGF, EDGF, VEGF, NT3, and NGF, BDNF, factor VIII, factor IX and insulin. For purposes of this invention, it is understood that “polypeptide” also includes molecules containing two or more polypeptide chains. Two or more polypeptide chains may be covalently linked, by way of non-limiting example, with respect to antibodies and insulin. Alternatively, the polypeptides may also be non-covalently associated, as occurs in multi-protein complexes.

Another class of molecules that can serve as transportable compounds according to the invention comprise nucleic acid molecules. Nucleic acid molecules are comprised of deoxyribonucleotides (i.e., deoxyribonucleic acids, DNA), ribonucleotides (i.e., ribonucleic acids, RNA), or combinations of deoxyribonucleotides and ribonucleotides. The nucleotides incorporated into such molecules can be naturally occurring (e.g., A, G, C, T, and U), or derivatives or modifications of naturally occurring nucleotides (e.g., ddA, ddG, ddC, ddT, ddI, AZT, and araG). Nucleic acids can be single- or double-stranded, or partially single- and partially double-stranded. Nucleic acid molecules include double-stranded molecules comprising at least one single-stranded DNA molecule complexed with at least one other single-stranded DNA or RNA molecule. Single-stranded nucleic acid molecules can form double-stranded duplexes by Watson-Crick base pairing over regions that are complementary, preferably completely complementary. Nucleic acid molecules can be produced biosynthetically, for example, in a cell or a cell extract. Alternatively, they can be chemically synthesized by any suitable method, preferably by a suitable solid state method. Those that are chemically synthesized may optionally include non-naturally occurring linkages between nucleotides so as to alter one or more properties of the molecule, for example, to render the resulting nucleic acid molecule resistant to enzymatic degradation in vivo.

Preferred nucleic acid molecules for inclusion in compositions according to the invention include plasmids and oligonucleotides. Plasmids are autonomous extrachromosomal circular double-stranded DNAs that are capable of being replicated by a cellular mechanism. Typically, a plasmid, in addition to an origin of replication, includes one or more genes that can be expressed. Such genes include those that encode selectable markers (e.g., antibiotic resistance genes and a gene coding for an auxotrophic marker), reporter genes (e.g., luciferase, green fluorescent protein), as well as genes that code for a biologically desired function, for example, a protein (e.g., an enzyme), antisense RNA, or ribozyme. Gene expression is under the control of one or more regulatory elements, for example, promoters, anti-termination sequences, termination of transcription signals, and polyadenylation sequences. Transcription of a particular gene is initiated at a promoter. The promoter may either be the naturally occurring promoter for the gene, or it may be from another source (including non-naturally occurring promoters derived by comparison of promoters having a desired activity, for example, high rates of transcription initiation). Preferably, promoters are inducible, so that expression of the corresponding gene can be activated when desired. Assembly of plasmids to incorporate the desired regulatory elements and genes is within the skill of those in the art using recombinant DNA techniques, and the particular regulatory elements and genes to be incorporated are left to the skilled artisan's discretion.

Other preferred nucleic acid molecules include oligonucleotides. Oligonucleotides are polymers of nucleotides assembled by synthetic chemical methods. Oligonucleotides typically comprise from about 8 to about 300 or more, preferably about 15 to about 100, nucleotides. Oligonucleotides are often used as single-strands, although complementary (completely or partially) oligonucleotides can be synthesized and assembled into double-stranded duplexes for inclusion into the compositions of the invention. Like the other nucleic acids of the invention, oligonucleotides can be comprised of deoxyribonucleotides, ribonucleotides, derivatives and modification thereof, as well as combinations thereof. Typical uses include antisense, ribozyme and triplex formation applications.

Lipids and carbohydrates represent other classes of transportable compounds. Preferred lipids and fatty acids include palmitic acid, lauric acid, stearic acid, palmitoleic acid, oleic acid, linoleic acid, linolenic acid, triglycerides, phosphoglycerides (including phosphatidylethanolamine, phosphatidylcholine, phosphatidylserine, phosphatidyllinositol), sphingolipids, sterols and their fatty acid esters. Carbohydrates include saccharides (which include sugars), heparin, heparin sulfate, and other heparin derivatives. Saccharides are divided into monosaccharides, disaccharides, trisaccharides, etc. or into oligosaccharides or polysaccharides, according to the number of saccharide groups (CₙH₂ₙOₙ₋₁, where n=1, 2, 3, 4, 5, 6, 7, 8, 10, or more) comprising the molecule. Transportable compounds also include those involving combinations of various compounds. For example, peptides and proteins may be glycosylated, as can lipids. Transportable compounds also include complexes of various compounds. For example, liposomes can be generated that comprise a one or more transportable proteins (of the same or different identities) incorporated into a lipid bilayer. Similarly, “gene delivery vehicles” can be generated to deliver nucleic acids encapsulated in a lipid coat. Any gene delivery vehicle can be employed, including viruses engineered to express one or more desired genes, so called “naked DNA expression vectors, as well as wholly synthetic structures that comprise an expression vector assembled into a lipid vesicle. Gene delivery vehicles also include proteonucleic acids (PNAs), where a nucleic acid molecule is complexed with positively charged peptides, proteins, or amino acid polymers (e.g., polylysine). In summary, in the context of this invention, a transportable compound is any compound that can be transported across a cell's plasma membrane as part of a transportable complex.

To form a transportable complex, the transportable compound is associated with a recognition element for a cell surface transport moiety. The association between the recognition element and the transportable compound can be covalent or non-covalent. Such covalent associations include those wherein the recognition element is directly covalently linked to the transportable compound, as well as those wherein a linker is employed. In this context, “direct covalent linkage” means that a covalent bond is formed between an atom of the recognition element and an atom of the transportable compound. Alternatively, the recognition element can be non-covalently associated with the transportable compound. Such non-covalent associations generally involve electrostatic interactions, van der Waals interactions, and/or steric interaction between one or more atoms of the associated molecules. As with covalent association, a non-covalent association can occur directly between atoms of the recognition element and atoms of transportable compound. Alternatively, the association can be mediated through one or more intermediary molecules. For example, an antibody engineered to bind the recognition element and the transportable compound can be used to associate the two molecules for purposes of this invention. Similarly, two antibodies, a first antibody specific for the recognition element and labeled with strepavidin (or any other member of a high affinity binding pair) and a second antibody specific for the transportable compound and labeled with biotin (or the corresponding other number of the particular high affinity binding pair), can be used to associate the recognition element and the transportable compound. As is known in the art, many other suitable configurations for non-covalent association can be generated and are within the scope of the invention.

A “recognition element” refers to any molecule that can recognize the desired cell surface transport moiety. Preferably, but not necessarily, the recognition involves a specific, moderate to high affinity, non-covalent association between the recognition element and the desired cell surface transport moiety, or a moiety associated with the cell surface transport moiety. Here, “specific” refers to at least a two fold, and preferably a 3-fold, 5-fold, 10-fold, 50-fold, 100-fold, 1000-fold, or more fold preference of the recognition element to associate with the desired cell surface transport moiety, or a moiety associated with the cell surface transport moiety under physiological conditions (i.e., those environmental conditions encountered in a given state) as compared to one or more other molecules present. These conditions include solvent type, pH, buffering capacity, salt concentration and type, and temperature, and may also include other or different promoters. As those in the art will appreciate, what constitutes “physiological conditions” will vary, for example, depending on whether an in vivo or ex vivo state is under consideration, the type of organism and its age, weight, health, sex, level of activity, metabolic state, etc. In any event, it is well within the skill of the ordinary artisan to define and determine what particular conditions exist for a given physiological state. As for affinity, “moderate affinity” association refers to an association wherein the association constant between the two molecules is at least about 10⁴ M to 10⁶ M. “High affinity” association refers to an association wherein the association constant between the two molecules is at least about 10⁷ M or 10⁸ M, and preferably about 10⁹ M, 10¹⁰ M, 10¹¹ M or more.

In some aspects of the invention, the recognition element is an antibody, antibody fragment(s), soluble T-cell receptor, or T-cell receptor fragment(s) that recognizes the cell surface transport moiety. With regard to antibodies, they can be polyclonal, monoclonal (i.e., they recognize the same epitope (a single antigenic determinant) on an antigen (here, the targeted cell surface transport moiety or another moiety associated therewith), and antibody derivatives including but not limited to single chain antibodies (sFv's). Monoclonal antibodies and antibodies fragments can be produced from a variety of animal cells, preferably from mammalian cells, e.g., murine and human cells. “Humanized” antibodies and antibody fragments are those which have been engineered to be more human, in terms of amino acid sequence, chemical modifications such as glycosylation, etc., in order to reduce the antigenicity of the antibody or antibody fragment.

Wild-type antibodies are comprised of four polypeptide chains, two identical heavy chains and two identical light chains, and they have two antigen binding domains. The antigen binding domains are directed to the same epitope of an antigen, and an antibody is thus capable of binding two antigen molecules at the same time. Each antigen binding domain is comprised of one light chain and one heavy chain, and the two chains are linked by a disulfide bridge formed between cystiene residues in the carboxy-terminal region of each chain, which is distal from the N-terminal region of each chain that constitutes its portion of the antigen binding domain. The molecule is further stabilized by disulfide bridges between the two heavy chains in an area known as the hinge region, at regions nearer the C-terminus of the heavy chains than the locations where the disulfide bridges between the heavy and light chains are made. The hinge region also provides flexibility for the antigen-binding portions of an antibody.

As those in the art appreciate, an antibody's antigen specificity is determined 10 by the variable regions located in the N-terminal regions of the light and heavy chains. The tremendous variability afforded by the genomic reorganization that can occur in the genes coding for these regions of these proteins makes possible the extraordinary number of discrete epitopes that can be recognized by antibodies. Well known recombinant and cell culture techniques enable the production of antibodies including non-naturally occurring antibody and T-cell receptor variants that retain just the desired antigen targeting capability of antibodies. These variants or derivatives include antibody and T-cell receptor fragments. Preferred fragments include Fab fragments (i.e., an antibody fragment that contains the antigen-binding domain and comprises a light chain and part of a heavy chain bridged by a disulfide bond), Fab′ (an antibody fragment containing a single anti-binding domain comprising an Fab and an additional portion of the heavy chain, up through the hinge region), Fab′)₂ (two Fab′ molecules joined by interchain disulfide bonds in the hinge regions of the heavy chains; the Fab′ molecules may be directed toward the same or different epitopes), a bispecific Fab (an Fab molecule having two antigen binding domains, each of which may be directed to a different epitope), sFv (the variable, antigen-binding determinative region of a single light and heavy chain of an antibody linked together by a chain of about 10 to about 25 amino acids), bispecific sFv, a disulfide-linked sFvm and an Fab diabody (two cross-paired, non-covalent dimers of sFvs; the sFvs may be directed toward the same or different epitopes). Antibodies may be produced by any suitable method, for example, in vivo (in the case of polyclonal antibodies raised against a particular antigen, or set of antigens), in cell culture (as is typically the case for monoclonal antibodies, wherein hybridoma cells expressing the desired antibody are cultured under appropriate conditions), or in recombinat DNA expression systems (in the case of sFv's). Antibody fragments can be produced by any suitable method, as well. Preferred methods include expression in bacteria, insect, yeast or mammalian cells, engineered to produce high levels of a particular gene product.

In other aspects of the invention, the recognition element can comprise a ligand for the cell surface transport moiety. The ligand may be a naturally occurring ligand for the cell surface transport moiety, or it may be a synthetic ligand. Ligands include small molecules, peptides, proteins, nucleic acids and derivatives. Naturally occurring and synthetic ligands can be identified by suitable screening methods, preferably by high throughput screening (HTS) methods, where 10, 50, 100, 1000, or more different compounds can be simultaneously assayed. Such methods are preferably performed in vitro. Sources for compounds to be screened include natural product extract libraries, libraries of existing known chemical compounds, and libraries of chemical compounds produced by combinatorial chemistry methods.

A transportable complex also comprises a cell surface transport moiety. These are molecules, typically proteins, expressed or otherwise presented on the exterior surface of a cell's plasma membrane that are involved in the transport of molecules across the plasma membrane or through the cell, and preferably transport molecules that contact the exterior of the cell at one location (e.g., the lumenal or exposed surface of an epithelial cell (namely that surface not in prolonged contact with other cells, except for migrating cells) to another location (e.g., the basolateral surface of an epithelial cell) so it can be released from the cell. A variety of protein receptors expressed on the surface of one or more cell types are known to be involved in transporting molecules across the cell membrane and through the cell. Examples include transferrin receptor, low density lipoprotein (LDL) receptor, and fibroblast growth factor receptors (FGFRs) and polymeric immunoglobulin receptors. The invention envisions targeting any of these moieties with a composition according to the invention. Given the importance of epithelium in the uptake and transport of compounds into and out of organisms, it is preferred to direct the compositions of the invention to cell surface transport moieties expressed on the surface of these cells, alone or as part of multi-protein complexes.

Preferred examples of such moieties include polymeric immunoglobulin receptors. A “polymeric immunoglobulin receptor” or “pIgR” is a class of receptor proteins expressed on or by cells such as those located in the respiratory tract, the gastrointestinal tract, the urinary and reproductive tracts, the nasal cavity, buccal cavity, ocular surfaces, dermal surfaces and any other mucosal epithelial cells. A particularly preferred pIgR is described in U.S. Pat. No. 6,042,833, although it is understood that, in the context of this invention, pIgR also refers to any of that receptor's family or superfamily members, any homologue of those receptors identified in other organisms, any splice variants of these receptors, as well as any fragments, derivatives, mutations, or other modifications expressed on or by cells such as those located in the respiratory tract, the gastrointestinal tract, the urinary and reproductive tracts, the nasal cavity, buccal cavity, ocular surfaces, dermal surfaces and any other mucosal epithelial cells. It is also to be understood that terms “secretory component membrane,” “secretory component membrane bound,” and “secretory component transmembrane” are equivalent to what is called “polymeric immunoglobulin receptor” or “pIgR” herein.

The compositions of the invention can further comprise other chemical components, such as diluents and excipients. A “diluent” is a chemical compound diluted in a solvent, preferably an aqueous solvent, that facilitates dissolution of the transportable complex in the solvent, and it may also serve to stabilize the biologically active form of the transportable complex or one or more of its components. Salts dissolved in buffered solutions are utilized as diluents in the art. For example, preferred diluents are buffered solutions containing one or more different salts. A preferred buffered solution is phosphate buffered saline (particularly in conjunction with compositions intended for pharmaceutical administration), as it mimics the salt conditions of human blood. Since buffer salts can control the pH of a solution at low concentrations, a buffered diluent rarely modifies the biological activity of a transportable compound.

An “excipient” is any more or less inert substance that can be added to a composition in order to confer a suitable property, for example, a suitable consistency or to form a drug. Suitable excipients and carriers include, in particular, fillers such as sugars, including lactose, sucrose, mannitol, or sorbitol cellulose preparations such as, for example, maize starch, wheat starch, rice starch, agar, pectin, xanthan gum, guar gum, locast bean gum, hyaluronic acid, casein potato starch, gelatin, gum tragacanth, methyl cellulose, hydroxypropylmethyl-cellulose, polyacrylate, sodium carboxymethylcellulose, and/or polyvinylpyrrolidone (PVP). If desired, disintegrating agents can also be included, such as cross-linked polyvinyl pyrrolidone, agar, or alginic acid or a salt thereof such as sodium alginate. Other suitable excipients and carriers include hydrogels, gellable hydrocolloids, and chitosan. Chitosan microspheres and microcapsules can be used as carriers. See, by way of non-limiting example, WO 98/52547 (which is stated to describe microsphere formulations for targeting compounds to the stomach, the formulations comprising an inner core and optionally including a gelled hydrocolloid) containing one or more active ingredients, a membrane comprised of a water insoluble polymer (e.g., ethylcellulose) to control the release rate of the active ingredient(s), and an outer layer comprised of a bioadhesive cationic polymer, for example, a cationic polysaccharide, a cationic protein, and/or a synthetic cationic polymer; U.S. Pat. No. 4,895,724. Typically, chitosan is cross-linked using a suitable agent, for example, glutaraldehyde, glyoxal, epichlorohydrin, and succinaldehyde. Compositions employing chitosan as a carrier can be formulated into a variety of dosage forms, including pills, tablets, microparticles, and microspheres, including those providing for controlled release of the active ingredient(s). Other suitable bioadhesive cationic polymers include acidic gelatin, polygalactosamine, polyamino acids such as polylysine, polyhistidine, polyornithine, polyquaternary compounds, prolamine, polyimine, diethylaminoethyldextran (DEAE), DEAE-imine, DEAE-methacrylate, DEAE-acrylamide, DEAE-dextran, DEAE-cellulose, poly-p-aminostyrene, polyoxethane, copolymethacrylates, polyamidoamines, cationic starches, polyvinylpyridine, and polythiodiethylaminomethylethylene.

The compositions of the invention can be formulated in any suitable manner. The transportable complexes therein may be uniformly (homogeneously) or non-uniformly (heterogenously) dispersed in the carrier. Suitable formulations include dry and liquid formulations. Dry formulations include freeze dried and lyophilized powders, which are particularly well suited for aerosol delivery to the sinuses or lung, or for long term storage followed by reconstitution in a suitable diluent prior to administration. Other preferred dry formulations include those wherein a composition according to the invention is compressed into tablet or pill form suitable for oral administration or compounded into a sustained release formulation. When the composition is intended for oral administration but the transportable complex is to be delivered to epithelium in the intestines, it is preferred that the formulation be encapsulated with an enteric coating to protect the formulation and prevent premature release of the transportable complexes included therein. As those in the art will appreciate, the compositions of the invention can be placed into any suitable dosage form. Pills and tablets represent some of such dosage forms. The compositions can also be encapsulated into any suitable capsule or other coating material, for example, by compression, dipping, pan coating, spray drying, etc. Suitable capsules include those made from gelatin and starch. In turn, such capsules can be coated with one or more additional materials, for example, and enteric coating, if desired. Liquid formulations include aqueous formulations, gels, and emulsions.

Some aspects of the invention concern compositions that comprise a bioadhesive, preferably a mucoadhesive, coating. A “bioadhesive coating” is a coating that allows a substance (e.g., a composition or transportable complex according to the invention) to adhere to a biological surface or substance better than occurs absent the coating. A “mucoadhesive coating” is a preferred bioadhesive coating that allows a substance, for example, a composition according to the invention, to adhere better to mucosa occurs absent the coating. For example, micronized particles (e.g., particles having a mean diameter of about 5, 10, 25, 50, or 100 μm) can be coated with a mucoadhesive. The coated particles can then be assembled into a dosage form suitable for delivery to an organism. Preferably, and depending upon the location where the cell surface transport moiety to be targeted is expressed, the dosage form is then coated with another coating to protect the formulation until it reaches the desired location, where the mucoadhesive enables the formulation to be retained while the transportable complexes interact with the target cell surface transport moiety.

The invention's compositions facilitate administration of transportable complexes to an organism, preferably an animal, preferably a mammal, bird, fish, insect, or arachnid. Preferred mammals include bovine, canine, equine, feline, ovine, and porcine animals, and non-human primates. Humans are particularly preferred. Multiple techniques of administering or delivering a compound exist in the art including, but not limited to, oral, aerosol (e.g., for nasal or pulmonary delivery), parenteral, and topical administration. Preferably, sufficient quantities of the transportable compound are delivered to achieve the intended effect. The particular amount of transportable compound to be delivered will depend on many factors, including the effect to be achieved, the type of organism to which the composition is delivered, delivery route, dosage regimen, and the age, health, and sex of the organism. As such, the particular dosage of transportable complex included in a given formulation is left to the ordinarily skilled artisan's discretion.

Thus, another aspect of the invention relates to delivering a composition according to the invention to an organism. As a result, the transportable complex of the composition is delivered to cells expressing the desired cell surface transport moiety.

A related aspect concerns various applications for the compositions of the invention. These include prophylactic and therapeutic applications. A non-limiting example of a prophylactic application is vaccination, wherein a composition according to the invention allows an antigen, presented as the transportable compound, to be delivered and elicit an immune response, preferably a protective immune response, in the organism to which the composition was administered. In a therapeutic context, the compositions allow a transportable compound having a therapeutic effect to be efficaciously delivered as part of a transportable complex. Because transportable complexes are delivered into cells by active transport, the instant compositions afford better control over bioavailability of transportable compounds, as compared to passive transport mechanisms. As such, the compositions of the invention enable improved uptake and utilization of the transportable compound.

### DETAILED DESCRIPTION OF THE INVENTION

The goal of delivering pharmaceutical compounds, particularly large molecular weight compounds, to site-specific targets has led to intensive research in this area. Numerous delivery pathways have been investigated, described, and utilized including alternatives to conventional delivery methods such as oral ingestion of pills and tablets and subcutaneous injection. Some of these methods include dermal, nasal, pulmonary, buccal, ocular, vaginal, and rectal administration as well as oral administration by inhalation and in liquid forms. Alternative delivery methods such as these have numerous advantages of their conventional counterparts. For example, nasal and/or inhalation delivery is particularly effective for individuals who have a fear of needles or have difficulty swallowing pills.

Inhalation therapy provides an attractive route of administration of such agents. Dry or liquid particles can be prepared and inhaled with the aid of dry-powder dispersers, liquid-aerosol generators or nebulizers. Inhalation and nasal delivery provides several advantages over more traditional means. The nasal and respiratory tract has a large surface area and high permeability compared to the gastrointestinal tract. The subepithelial layer is highly vascularized and sustained delivery is possible. Nasal inhalation also affords the opportunity to avoid the loss of a substantial portion of the agent by the first pass effect. In addition, synthesis of more stable lipophillic peptide analogues, use of pepitase and protease inhibitors, the application of absorption enhancers, and the use of different formulations such as sprays, drops, and viscous agents can further enhance delivery pathway in the nasal/inhalation pathway.

Other mucous membranes can be targeted as well. For example, calcitonin has been delivered by the vaginal route following encapsulation into esterified hyaluronic acid (Hyaff™) microspheres (see Bonucci, et al. Calcif. Tissue Int. 56, 274-279 (1995)). See also WO98/47535. In many cases, a synthetic polymer, device, or carrier system is used with the appropriate properties to target a specific site within the body. The invention disclosed herein is contemplated for use with such alternative delivery systems, several of which are described in detail below, as well as more traditional pathways. When targeting mucous membranes, it is preferred to include a mucoadhesive coating on the composition.

Numerous methods for protecting the compound to be delivered and enhancing the retention and absorption during its delivery are also well-known in the art (see, e.g., U.S. Pat. No. 4,675,189). For example, controlled-release tablets and capsules, especially those with enteric coatings (see, e.g., U.S. Pat. No. 5,958,455 and references contained therein) allow the effective compound to be delivered to specific portion of the gastrointestinal tract based on known breakdown times for the given coating and matrix. Liquid applications which gel upon administration (such as those discussed in WO98/47535 and references contained therein) allow the compound delivered increased duration and thus delivery time at the site-specific target. The addition of enzyme inhibitors such as protease inhibitors (such as those cited in U.S. Pat. No. 6,042,833 and references cited therein) to a compound increases the duration which the compound remains intact through the gastric system. Finally, encapsulation in microparticles and mucoadhesives improves compound delivery efficiency by enhancing targeting of site-specific in tissues and cells and increasing retention within those tissues and cells. U.S. Pat. No. 5,985,309 provides a thorough review of microparticle formation and utilization in addition to the discussion below. Muscoadhesives known in the art include, but are not limited to, pectin, biotin, chitosan, polycarbophil, polysaccharides, lipopolysaccharides, oligosaccharides, acrylic acid, methacrylic acid, alginic acid, hyaluronic acid, gum tragacanth, karaya gum, and carboxymethylcellulose, hydroxypropylmethyl cellulose, hydroxypropyl cellulose, carbomer, polycarbophil, as well as those disclosed in U.S. Pat. Nos. 5,744,155, 5,989,535, WO97/20576, WO98/01161, WO98/47535, WO98/52547, and references cited therein, and mixtures of any of the foregoing.

The microparticles utilized by inhalation systems generally range in size from about 1 μm to about 25 μm (mean volume diameter), although inhaled particles more than 10 μm in diameter may be trapped in the nasal passages, throat, larynx, and bronchial walls. Particles less than about 5 μm in diameter may penetrate more deeply into the alevoli of the lung. Once deposited in the lung, the alveoli provide a large surface area for rapid transfer into the pulmonary circulation.

Certain types of microparticles can enhance the effect of inhalation therapy delivery, retention, and uptake. Attempts to develop sustained-release formulations have included the use of a variety of biodegradable and non-biodegradable polymer (e.g. poly(lactide-co-glycolide)) microparticles containing the active ingredient (see e.g., Wise et al., Contraception, 8:227-234 (1973); and Hutchinson et al., Biochem. Soc. Trans., 13:520-523 (1985)), and a variety of techniques are known by which active agents, e.g. proteins, can be incorporated into polymeric microspheres (see e.g., U.S. Pat. No. 4,675,189 and references cited therein). Other formulations may be in solution or in the form of a spray-dried powder. Spray-dried powders can be prepared using procedures known by those skilled in the art; see e.g., Masters, K., “Spray Drying Handbooks” (John Wiley & Sons, eds., New York 1984) and U.S. Pat. No. 6,022,737 and references cited therein including Broadhead et al., Drug Dev. and Indust. Pharmacy 18:1169-1206 (1992).

Thus, in one example of the disclosed in invention, microparticles are the dosage form of choice for the transportable complex and can be prepared in a number of ways. For example, in the aforementioned spray-drying method, the polymer and composition are mixed together in a solvent for the polymer, and then the solvent is evaporated by spraying the solution, leaving polymeric droplets containing the transportable composition.

A preferred method for producing microspheres comprising compositions according to the invention suitable for pulmonary delivery involves spray drying a soluble mixture comprising a transportable complex and a soluble polysaccharide. Here, “microsphere” preferably means microparticles of a substantially spherical nature, with those having substantially granular and/or non-spherical natures being less preferred. Here, “substantially” means greater than about 50%, preferably greater than about 80%, and even more preferably greater than about 90%, spherical, granular, or non-spherical, as the case may be. The microspheres produced by this process are appropriately sized for depositing a composition according to the invention into the alveoli. It is known that solid particles intended for pulmonary delivery should have an aerodynamic diameter (see “Aerosols in Medicine,” Moren, et al. (1993), Elsevier)) of less than about 10 μm, preferably less than about 5 μm, although the particles should not be too small, or they will fail to be deposited or be exhaled. Generally, the particles should be about 0.25 to about 10 μm in diameter, with sizes ranging from 0.5 to 5 μm being preferred. Also, because some transportable compounds, e.g., peptides and proteins, must be administered in small dosages (e.g., less than 1 milligram (mg)), they should mixed with an inert carrier, for example, lactose and/or mannitol (see, by way of non-limiting example, WO 95/31479).

The polysaccharides used in this process preferably exclude disaccharides, e.g., lactose, generally have a molecular weight ranging from between about 10,000 to about 1,000,000, preferably between about 50,000 and 750,000, particularly between about 100,000 and 300,000, and are water soluble (which in this context, means can be dissolved in an aqueous solution at a concentration of about 1 mg/ml or greater). Suitable soluble polysaccharides that can be used in such formulations include amylodextrin, amylopectin, hydroxyethylstarch, carboxymethylcellulose, diethylaminotethyldextran, dextran, pullulan, carboxymethyl pullulan, and polyglucosamine. Mucopolysaccharides, e.g., hyaluronic acid, can also be used. In addition, the formulation may be spray dried from an emulsion containing the polysaccharide and a transportable complex. Emulsions are known in the art, and any suitable emulsion system may be used. For example, see WO 98/52547. Of course, as with the other formulations of the invention, more than one polysaccharide (or other carrier) may be employed in a given formulation, and the formulation may also comprise more than one specie of transportable complex.

Briefly, one method for producing microspheres comprising a polysaccharide and a transportable complex involves dissolving the transportable complex in a suitable solvent (e.g., water or phosphate buffered saline). The amount of transportable complex dissolved will depend on the dose of the transportable compound required in the final dosage form. Typically, the amount will range from about 100 μg to 5 mg, although other amounts may be used, depending on the particular circumstance. The desired polysaccharide(s) is(are) also dissolved, preferably in the same solvent type. The amount of polysaccharide dissolved depends on its gelation and rheological properties, and typically ranges from about 0.01 g to about 50 g in 200 ml, preferably 0.1 g to about 20 g in 200 ml, with about 1 g in 20 ml being preferred. When charged polysaccharides are used, pH and ionic strength can be adjusted as needed.

The two solutions are then combined. Preferred final polysaccharide concentrations are in the range of from about 0.5 g to about 5 g per 20 ml, especially about 0.75 g to about 3 g per 20 ml. In any event, the viscosity of the resultant solution should be suitable for dispersion in the particular spray drying device to be used, although a viscosity in the range of about 1 to about 15 centipoise is generally preferred. Any suitable spray drying device can be used to produce particles having the desired size (see, by way of non-limiting example, WO 97/35562). Also, variations in polysaccharide concentration and processing conditions, such as cross-linking degree or the addition of starch gel modifiers (e.g., fatty acids such as myrisate and monoglycerides), can produce microparticles of different sizes and having different release characteristics, for example, rapid or sustained release of the transportable complex from the microsphere. Those in the art will understand that other excipients may also be included in such formulations, for example, to provide controlled release of the transportable complexes contained in the microspheres. Representative excipients include phospholipids, cyclodextrins, gelatin, and alginate. Cross-linking agents can also be used to confer controlled release properties on the microspheres, although it desirable that any cross-linked molecules so produced be completely biodegradable. Polyphosphates are preferred for use in this regard with polysaccharides, and aldose sugars are preferred for use with polyglucosamines.

The above formulations may be delivered to the lung in any suitable fashion, including pulsitile and controlled fashions. Devices such as metered dose inhalers (MDIs), which typically employ a volatile propellant such as a CFC liquid or a non-CFC alternative, blister packs, and other dry powder devices.

Transportable compounds suitable for delivery in this way include anti-asthma compounds, peptides, proteins, small molecules, nucleic acids. These compounds can be delivered for local or systemic effect, as required.

Generally, such microsphere-containing formulations are administered to the lung of a human in quantities ranging from about 0.1 to about 500 mg, preferably about 1 to 100 mg, particularly about 5 to 50 mg. The transportable compound content of the formulation may range from less than about 0.01% w/w of the formulation to more than about 50%. Of course, the level of loading will depend on the particular transportable compound, its specific activity, the intended delivery location, the intended application (e.g., the intended therapeutic or prophylactic indication), the intended distribution of the transportable compound (local, systemic), the properties of the particular microspheres, the device to be used, etc.

Another technique which can be used to form microspheres is solvent evaporation. Solvent evaporation involves the dissolving of the polymer in an organic solvent which contains either dissolved or dispersed active ingredient. The polymer/active ingredient mixture is then added to an agitated continuous phase which is typically aqueous. Emulsifiers are included in the aqueous phase to stabilize the oil-in-water emulsion. The organic solvent is then evaporated over a period of several hours or more, thereby depositing the polymer around the core material. For a complete review of the solvent evaporation procedure (see, by way of non-limiting example, U.S. Pat. No. 4,389,330, and references cited therein).

Yet another technique which can be used to form microspheres is phase separation, which involves the formation of a water-in-oil emulsion or oil-in-water emulsion. The polymer is precipitated from the continuous phase onto the active agent by a change in temperature, pH, ionic strength or the addition of precipitants. For a review of phase separation techniques, see, e.g., U.S. Pat. No. 4,675,800 and references cited therein.

The release characteristics of the transportable composition from microparticles prepared by methods such as those described above may be continuous or discontinuous, and in some cases, the initial level of active ingredient release is too high or too low. Thus, various additives are often utilized in an attempt to control the release of the transportable compound or complex (see, e.g., EP 0 761 211 A1).

To avoid the denaturation of protein and other fragile biological molecules which occurs upon spray drying, solvent evaporation or phase separation by classical techniques, the emulsion of polymers and active ingredient can be atomized into frozen nonsolvent overlayed with liquified gas such as nitrogen to form particles, and then extracted at very low temperatures. The low processing temperatures may preserve the activity and integrity of the fragile biological molecules such as proteins.

A transportable composition contained within polymeric microparticles can be created in which the mixture of the active ingredient and the polymer are dispersed within a continuous phase, the resulting dispersion is frozen, and the water and organic solvents removed from the dispersion by lyophilization. Further, a composition for the sustained-release of the transportable composition comprising a biologically active ingredient contained within polymeric microparticles, or, alternatively, a biologically active ingredient loaded onto the polymeric microparticles can be created. The sustained-release compositions of the present invention maintain the activity and integrity of the active ingredient during encapsulation and release, which helps to provide for longer periods of consistent release.

The foregoing microparticle preparation techniques are representative, rather than exhaustive, of those known to one of ordinary skill in the art. Additional methods, such as the preparation of protein-loaded poly(Σ-caprolactone) microparticles discussed in M. -A. Benoit et al. Int'l J. Pharm. 184, 73-84 (1999), may be also practiced in the preparation of a suitable carrier. A review of microparticle preparation appears in U.S. Pat. No. 5,985,309 and references cited therein.

In other aspects of the invention, capsules may serve as the dosage form of choice for the transportable complex. The carrier and transportable complex composition may be in a number of forms including dry powder, particulate, aqueous solution, oil-in-water emulsion, or others known in the art. Capsule formulation may be for simple release of the complex or in a controlled release system. Furthermore, the carrier-complex contained in the capsule can be further enhanced by aforementioned means such as the inclusion of mucoadhesives, protease inhibitors, or target-specific microparticles with the transportable complex.

Numerous capsule manufacturing, filling, and sealing systems are well-known in the art. Preferred capsule dosage forms can be prepared from gelatin and starch. Gelatin has been the traditional material, and the dosage forms are generally produced by well known dip molding techniques. After manufacture, gelatin capsules are filled with the desired composition and then sealed. A more recently developed alternative to gelatin dosage forms are capsules produced from starch. Starch capsules (typically made from potato starch) afford several advantages compared to gelatin capsules, including pH-independent dissolution, better suitability for enteric coating, water in the dosage form is tightly bound to the starch (and is thus less likely to migrate into the composition encapsulated in the dosage form), and the absence of animal-derived ingredients (which may be antigenic or contaminated with pathogens, Vilivilam et al. (2000), PSTT, vol. 3, no. 2:64-69). Starch capsules are odorless and rigid, and exhibit similar dissolution properties as compared to gelatin capsules.

Capsules of any suitable size can be manufactured. Starch capsules are typically made in two pieces, a cap and a body, using injection molding techniques. See Eith et al. (1987), Manuf. Chem., vol. 58: 21-25; Idrissi et al. (1991), Pharm. Acta. Helv., vol. 66: 246-252; and Eith et al. (1986), Drug Dev. Ind. Pharm., vol. 12: 2113-2126. The two pieces are then sealed together during filling the prevent separation. Sealing can achieved by applying a hydroalcoholic solution to the inner surface of the cap.

After making the capsule dosage forms, if desired, they can be coated with one or more suitable materials. For example, when it is desired to deliver the encapsulated composition to the gastrointestinal tract, one or enteric coatings may be applied. Traditionally, enteric coatings were used to prevent gastric irritation, nausea, or to prevent the active ingredient from being destroyed by acid or gastric enzymes. These coatings can also be used to target deliver to particular gastrointestinal regions. A variety of enteric coatings are known in the art, and any suitable coating, or combinations of coatings, may be employed. Suitable coatings for starch capsules include aqueous dispersions of methacrylic acid copolymers and water-based reconstituted dispersion of cellulose acetate phthalate (CAP). See, by way of non-limiting example, Brogmann et al. (1994), Pharm. Res., vol. 11, S-167; Vilivalam et al. (1997), Pharm. Res., vol. 14, S-659; Vilivalam et. al. (1998), Pharm. Res. 15, S-645; Bums et al. (1996), Int. J. Pharm., vol. 134: 223-230; and Davis et al. (1992), Eur. J. Nucl. Med., vol. 19,: 971-986. A variety of coatings can be used to coat encapsulated dosage forms. These coatings include pH-sensitive materials, redox-sensitive materials, and materials that can be broken down by specific enzymes or microorganisms present in the intestine. Watts et al., WIPO publications WO 97/05093 and 98/15265, describe an enteric-coated starch capsule system for targeting sites in the colon. The pH sensitive enteric coating begins to dissolve when the dosage form enters the small intestine, and coating thickness dictates in which region of the intestine the capsule disintegrates, for example, in the terminal ileum or in the ascending, transverse, or descending colon. Other coatings, or combinations of coatings, can also be used to achieve the same effect.

After a dosage from is prepared, it is typically packaged in a suitable material. For pill or tablet dosage forms, the dosage forms may be packaged individually or bottled en masse. An example of individual packaging PVC-PVdC-Alu, where aluminum blisters are covered with PVC (polyvinyl chloride) coated with PVdC (polyvinylidene chloride) to improve water vapor and oxygen protection. Suitable bottling materials include tinted, transluscent, or opaque high density polyethylene.

Delivery of biologically active compounds via liposomes is also known in the art (see, by way of non-limiting example, U.S. Pat. No. 5,762,904 and references cited therein. Liposomes are typically less than about 10 microns in diameter and can be absorbed through the Peyer's patches having passed through the gastrointestinal tract. Liposomes also have some features that should be advantageous for a particulate system for macromolecule delivery. The phospholipid bilayer membrane of liposomes separates and protects entrapped materials in the inner aqueous core from the outside. Both water-soluble and -insoluble substances can be entrapped in different compartments, the aqueous core and bilayer membrane, respectively, of the same liposome. Chemical and physical interaction of these substances can be eliminated because the substances are in these different compartments. Liposomes can be prepared by a thin film hydration technique followed by a few freeze-thaw cycles or according to methods known to those skilled in the art, for example, as described in U.S. Pat. No. 4,522,811, incorporated herein by reference.

Delivery of macromolecules, specifically high molecular weight polypeptides, through the eye has been disclosed and represents another aspect of the invention. (See e.g. U.S. Pat. Nos. 5,182,258, 5,283,236, and 5,278,142 and references cited therein.) High molecular weight polypeptides such as those disclosed in the Chiou references are contemplated as a part of the transportable complex disclosed herein.

Similarly, mouthwashes and other non-traditional oral delivery mechanisms may be employed with this invention, for example, in the method disclosed by Beggs et al. in U.S. Pat. No. 5,490,988.

One skilled in the art would readily appreciate that the present invention is well adapted to carry out the objects and obtain the ends and advantages mentioned, as well as those inherent therein. The compositions and transportable complexes and the methods, procedures, treatments, molecules, and specific compounds described herein are presently representative of certain aspects of the invention, and thus are exemplary and are not intended as limitations on the scope of the invention. Alternatives, equivalents, changes, and other uses will occur to those skilled in the art, and those are encompassed within the spirit of the invention are defined by the scope of the claims below.

It will be readily apparent to one skilled in the art that varying substitutions and modifications may be made to the invention disclosed herein without departing from the scope and spirit of the invention.

All patents and publications mentioned in the specification are indicative of the levels of those skilled in the art to which the invention pertains, and any and all such patents and publications are hereby incorporated by reference to the same extent as if each individual patent and publication was specifically and individually indicated to be incorporated by reference.

The invention illustratively described herein suitably may be practiced in the absence of any element or elements, limitation or limitations which is not specifically disclosed herein. Thus, for example, in each instance herein any of the terms “comprising,” “consisting essentially of,” and “consisting of” may be replaced with either of the other two terms. The terms and expressions that have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed herein, optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups, those skilled in the art will recognize that the invention is also thereby described in terms of any individual member or subgroup of members of the Markush group.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other aspects of the invention are within the following claims.

1. A composition comprising a carrier and a transportable compound wherein the transportable compound is associated with at least one recognition element for a cell surface transport moiety, wherein the cell surface transport moiety is involved in the transport of molecules across a plasma membrane or through a cell.

2. A composition according to claim 1 wherein the association between the recognition element and the transportable compound is covalent.

3. A composition according to claim 1 wherein the association between the recognition element and the transportable compound is non-covalent.

4. A composition comprising a carrier and a transportable complex comprising at least one transportable compound, wherein the transportable complex is associated with a recognition element for a cell surface transport moiety, wherein the cell surface transport moiety is involved in the transport of molecules across a plasma membrane or through a cell.

5. A composition according to claim 4 wherein the association between the recognition element and the transportable complex is covalent.

6. A composition according to claim 4 wherein the association between the recognition element and the transportable complex is non-covalent.

7. A composition according to claim 1 or claim 4 further comprising at least one protease inhibitor.

8. The composition of claim 1 or claim 4 wherein said transportable compound is selected from the group consisting of a small molecule, a protein, a peptide, a polypeptide, a nucleic acid, a lipid, a carbohydrate, a fatty acid, a polysaccharide, and an antigen.

9. The composition of claim 4 wherein the transportable complex is a liposome.

10. The composition of claim 4 wherein the transportable complex is a gene delivery vehicle.

11. A composition according to claim 1 or claim 4 wherein the recognition element is a small molecule.

12. A composition according to claim 1 or claim 4 wherein the recognition element is a polypeptide.

13. A composition according to claim 1 or claim 4 wherein the recognition element is an antibody or an antibody fragment.

14. A composition according to claim 13 wherein the antibody is a single chain variable region antibody fragment.

15. A composition according to claim 13 wherein the antibody or antibody fragment is specific for an epitope located on a stalk portion of a polymeric immunoglobulin receptor.

16. A composition according to claim 13 wherein the antibody or antibody fragment is specific for an epitope located on a secretory component a polymeric immunoglobulin receptor.

17. A composition according to claim 1 or claim 4 wherein the recognition element is a ligand for a cell surface transport moiety, wherein the cell surface transport moiety is involved in the transport of molecules across a plasma membrane or through a cell.

18. The composition of claim 17, wherein the ligand is a naturally occurring ligand.

19. The composition of claim 17, wherein the ligand is a synthetic ligand.

20. The composition of claim 17, wherein the ligand is selected from the group consisting of a small molecule, a peptide, a protein, a nucleic acid and derivatives thereof.

21. A composition according to claim 1 or claim 4 wherein the cell surface transport moiety is a receptor involved in the transport of molecules across a plasma membrane or through a cell.

22. The composition of claim 21 wherein the receptor is selected from the group consisting of a transferrin receptor, a fibroblast growth receptor, a low density lipoprotein receptor and a polymeric immunoglobulin receptor.

23. A composition according to claim 21 wherein the cell surface transport moiety is a polymeric immunoglobulin receptor.

24. A composition according to claim 21 wherein the cell surface transport moiety is a secretory component of a polymeric immunoglobulin receptor.

25. A composition according to claim 21 wherein the cell surface transport moiety is a stalk of a polymeric immunoglobulin receptor.

26. A composition according to claim 1 or claim 4 wherein the cell is an epithelial cell.

27. A composition according to claim 26 wherein the cell surface transport moiety is presented on the luminal surface of the cell.

28. A composition according to claim 1 or claim 4 wherein the carrier is a pharmaceutically acceptable carrier.

29. A composition according to claim 1 or claim 4 that is a composition selected from the group consisting of a dry composition, a liquid composition and an aqueous composition.

30. A composition according to claim 1 or claim 4 that is a mucoadhesive formulation.

31. A composition according to claim 1 or claim 4 encapsulated in an enteric coating.

32. A composition according to claim 1 or claim 4 that is a sustained release formulation.

33. A method for delivering a transportable compound to a cell, the method comprising exposing a cell to a composition according to claim 1 or claim 4.

34. A method according to claim 33 wherein the exposing occurs in vitro.

35. A method according to claim 33 wherein the exposing occurs in vivo.

36. A method according to claim 33 wherein at least one of the transportable compound and the transportable complex of the composition is transported across a plasma membrane.

37. A method according to claim 33 wherein at least one of the transportable compound and the transportable complex of the composition is moved across the cell.

38. A method according to claim 33 wherein at least one of the transportable compound and the transportable complex of the composition is moved through the cell.

39. A method according to claim 33 wherein at least one of the transportable compound and the transportable complex of the composition is released from a cell at a different location than the location at which it is contacted to the cell.

40. A method according to claim 39 wherein one of said locations is the basolateral surface of an epithelial cell.

41. A method of making a composition according to claim 1 or claim 4 comprising combining a carrier with a transportable or a transportable complex.

42. A method according to claim 41 further comprising encapsulating the composition.

43. A method according to claim 42 further comprising coating the encapsulated composition.

44. A method according to claim 41 wherein the composition is selected from the group consisting of a dry composition, a liquid composition and an aqueous composition.

45. A method according to claim 41 wherein the composition is a sustained release formulation.

46. A method according to claim 41 wherein the composition is suitable for inhalation therapy.
